(19) United States
(12) Patent Application Publication
(10) Pub. No.: US 2013/0319417 A1
(43) Pub. Date: Dec. 5, 2013

(54) SLEEP APNEA BREATHING MASK

(71) Applicant: Jonathan H. Weinman, Stevenson, MD (US)

(72) Inventor: Jonathan H. Weinman, Stevenson, MD (US)

(21) Appl. No.: 13/484,455

(22) Filed: May 31, 2012

(51) Int. Cl. A61M 16/06; A61M 16/00

(52) U.S. Cl. CPC A61M 16/0683; A61M 16/0057
USPC 128/205.25

(57) ABSTRACT

A breathing mask consists of a housing having a chamber surrounding the user's nose. An air inlet includes a coupling to a hose connected to a source of pressurized air. To each side of the chamber, connections are provided for a strap going around the head of the user above the ears. At the top of the chamber a bracket emanates upwardly and terminates at a short horizontal piece that has a cushion underneath engaging the forehead of the user.

### BACKGROUND OF THE INVENTION

The present invention relates to an improved sleep apnea breathing mask. Obstructive sleep apnea (OSA) is a potentially life threatening condition in which the victim has frequent episodes in which they either stop breathing or breathe less efficiently while sleeping. Studies have shown that OSA is caused by a blockage of the airway that typically results from collapse and resultant closure of soft tissue located rearward in the throat during sleep. In an otherwise healthy patient, when such episodes occur, their brain signals the patient to arouse and resume breathing, but subsequent sleep is often less restful.

In 2005, at least 12 million Americans exhibited symptoms of OSA, a significant number. Those exhibiting symptoms of OSA often have other serious health concerns including high blood pressure, heart disease, diabetes, and propensity to suffer strokes.

People who have been diagnosed with having the symptoms of OSA are often prescribed a therapeutic regime involving the use of a Continuous Positive Airway Pressure (CPAP) device. Such a device operates by having the patient wear a specially designed mask that facilitates delivery of a constant flow of air through the wearer's nose, mouth, or both. In so doing, the CPAP device continuously pressurizes the airway of the wearer, thereby preventing the collapse of the airway that causes the OSA condition. Patient compliance with therapy is a significant problem as many patients find CPAP masks cumbersome, confining and unduly burdensome to wear.

In the prior art, numerous examples of breathing masks used in connection with a CPAP system are known. One such example is known as the Fisher Paykel Zest mask. The Fisher Paykel Zest mask is illustrated in FIGS. 1 and 2. As generally designated by the reference numeral 10, this mask includes an inlet 11, a chamber 13 which is fed air from the inlet 11, and support structure including a vertically disposed bracket 15 and a horizontally disposed fitting 17 that includes loops 19 and 21 as well as two forehead pads 23.

The chamber 13 also includes side appendages 25 and 27 that carry further loops 29 and 31, respectively.

A first strap 33 encircles the head 1 of the user beneath the ears 3 thereof, while a second strap 35 encircles the head 1 above the ears 3. As seen in FIG. 1, the straps 33 and 35 may be interconnected by a vertically disposed strap 37 that keeps the straps 33 and 35 in generally parallel alignment to preclude them from slipping below the head in the case of the strap 33 or above the head in the case of the strap 35.

The Fisher Paykel Zest mask illustrated in FIGS. 1 and 2 is extremely uncomfortable to wear for many users. With its straps above and below the ears, its interconnecting straps 37, and its large forehead support cushions, the user is well aware of the existence of the mask on their head and this necessarily makes it more difficult to wear the mask and fall asleep with it. Ultimately, this leads to lack of compliance with OSA therapy for many wearers.

Applicant is aware of the following prior art:

U.S. Patents

| | |
|---|---|
| 4,919,128 to Kopala et al. | 7,575,005 to Mumford et al. |

U.S. Pat. No. 5,042,478 to Kopala et al. U.S. Pat. No. 7,610,916 to Kwok et al.

Published Applications

US 2003/0005509 to Kelzer
US 2004/0211428 to Jones, Jr. et al.
US 2004/0216747 to Jones, Jr. et al.
US 2008/0053446 to Sleeper et al.
US 2008/0060653 to Hallett et al.
US 2008/0178875 to Henry
US 2008/0245364 to Patterson
US 2008/0264422 to Fishman
US 2008/0314388 to Brambilla et al.
US 2009/0065005 to Ades
US 2011/0048426 to Sleeper et al.
US 2011/0066061 to Colman et al.
US 2011/0146685 to Allan et al.
US 2011/0209708 to Rapoport
US 2011/0259340 to Witt et al.

Of the listed references, most of them disclose face masks removably affixed to the head of the user using multiple straps and having an extremely wide forehead cushion. Those that don't exhibit these characteristics are the following:

While Kelzer shows a single strap 44 going around the head above the ears of the user, it connects to the face mask using a bracket that has multiple connection points with the mask, both to either side of the nose and to either side of the forehead. Moreover, Kelzer fails to teach or suggest a horizontally disposed cushion.

Fishman discloses sleep apnea headgear including one embodiment (FIG. 11) in which a single strap goes around the head above the ears. However, that strap is at the uppermost extent of the face mask leading to the possibility that the lower portion of the face mask may become spaced from surrounding the nose. A spring biased locking mechanism 64 is intended to counteract this possibility, but renders the Fishman mask unwieldy and complex.

Ades discloses a face mask having a single strap at the upper extent of the mask. This location causes potential lifting off of the lower portion of the mask from a sealed surrounding relation to the nose of the wearer.

Witt et al. disclose a mask with a single strap that does not cover the nose of the user, but rather includes portions inserted within the nostrils of the user which can be prone to dislodgment and air leakage. As such, no forehead cushioning is provided.

The present invention was devised to overcome the deficiencies of this prior art and to provide a simplification of a face mask that requires only a single strap and a smaller forehead cushion. It is with these thoughts in mind that the present invention was developed.

### SUMMARY OF THE INVENTION

The present invention relates to an improved sleep apnea breathing mask. The present invention includes the following interrelated objects, aspects and features:

(1) In a first aspect, the inventive breathing mask consists of a housing having a chamber sized and configured to surround the nose of the user. The portion of the chamber facing and engaging the user's face may be made of a flexible material to adapt to facial configurations that may differ.

(2) Centrally located on the chamber is an air inlet that may preferably be L-shaped and includes a coupling allowing it to be coupled to a hose connected to a source of pressurized air.

(3) To each side of the chamber, connections are provided for a strap that goes around the head of the user and is preferably located above the ears of the user. This is the only strap that is used to hold the mask to the user's head.

(4) At the top of the chamber a bracket emanates upwardly and terminates at a short horizontal piece that has a cushion underneath. The cushion engages the forehead of the user and prevents further pivoting movement about a horizontal axis in the direction of the forehead. The cushion, along with the location of the strap at the center of rotation of the mask, hold the portion of the chamber facing the face of the user firmly against their face to seal the chamber and ensure that all of the air coming into the air inlet goes into the nose of the user and is breathed by the user.

(5) The inventive breathing mask drastically simplifies the structure as compared to prior art masks. It is more lightweight, more easily placed on the face of the user, and more easily removed from the head of the user. Often the user wishes to leave their bed at night for a variety of reasons including to urinate or defecate. When they do so, often, they would prefer not to be completely awakened so that they can more easily go back to sleep. As compared to prior art breathing masks, the simplicity of the present invention including the fact that it is less discernible when placed on the head of the user makes it more easy for the user to awaken, remove the mask, defecate and/or urinate, and then replace the mask and go back to sleep.

Accordingly, it is a first object of the present invention to provide an improved sleep apnea breathing mask that encourages usage and compliance with OSA therapy.

It is a further object of the present invention to provide such a mask in which only a single strap is employed to hold the mask onto the user's head.

It is a still further object of the present invention to provide such a mask in which the single strap extends over the ears of the wearer.

It is a yet further object of the present invention to provide such a mask in which a small forehead engaging cushion is attached to the mask chamber so that the mask may not pivot toward the forehead.

It is a still further object of the present invention to provide such a mask in which an L-shaped inlet is provided to allow releasable attachment to a source of pressurized air.

These and other objects, aspects, and features of the present invention will be better understood from the following detailed description of the preferred embodiment when read in conjunction with the appended drawing figures.

### BRIEF DESCRIPTION OF THE DRAWINGS

FIG. 1 shows a side perspective view of a prior art breathing mask.

FIG. 2 shows a front view of the prior art breathing mask of FIG. 1.

FIG. 3 shows a side perspective view of the present invention.

FIG. 4 shows a front side perspective view of the present invention with a wearer's head shown in phantom and with portions not shown to show detail.

FIG. 5 shows a left side view of the inventive breathing mask.

FIG. 6 shows a front view of the inventive breathing mask.

FIG. 7 shows a right side view of the inventive breathing mask as worn by a wearer.

FIG. 8 shows a rear view of the strap of the breathing mask as worn by a wearer.

FIG. 9 shows a perspective view from the front and top of the present invention.

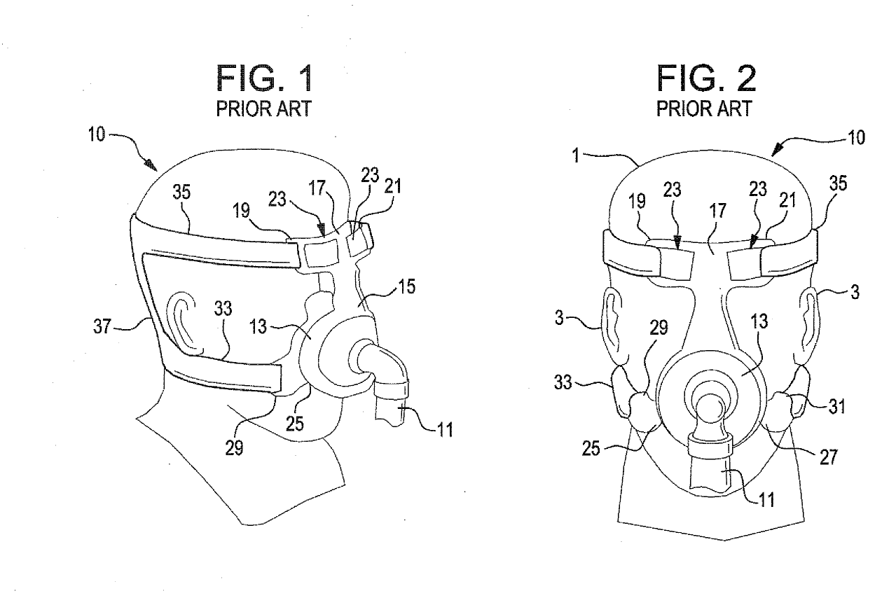

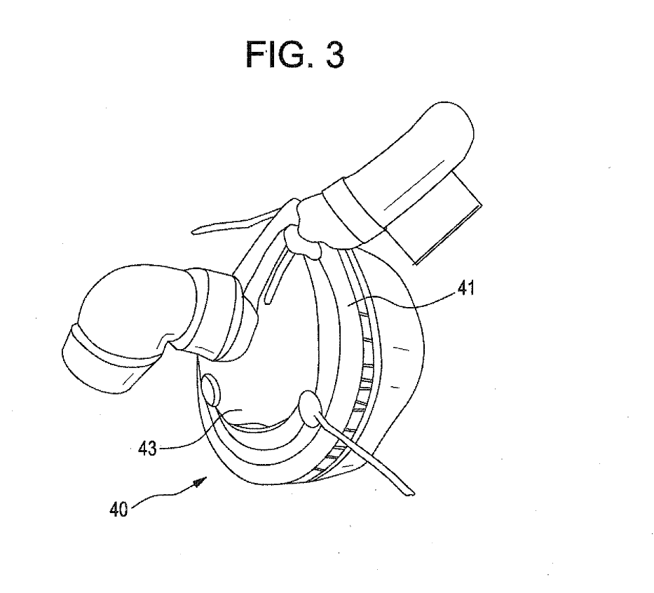

### SPECIFIC DESCRIPTION OF THE PREFERRED EMBODIMENT

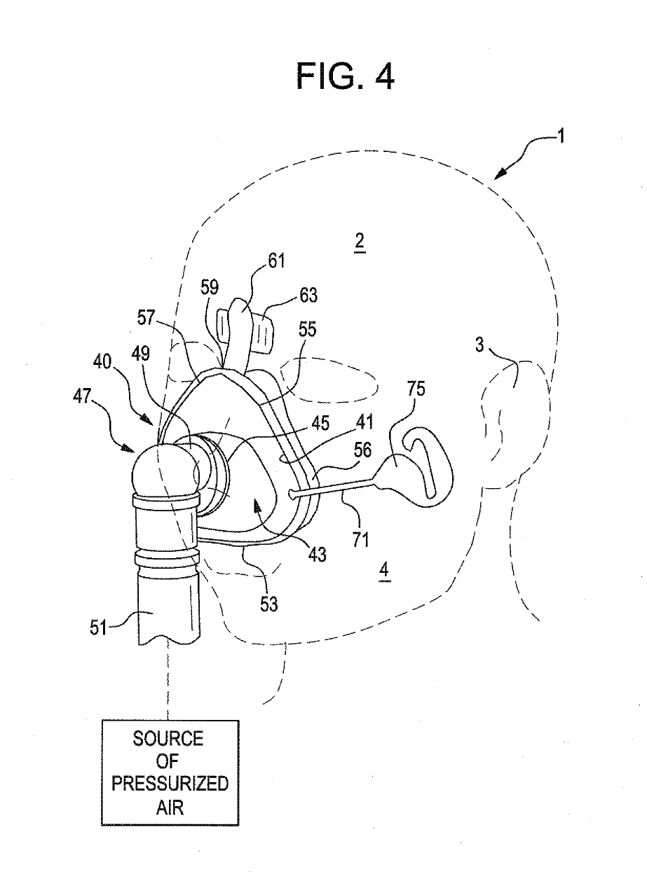

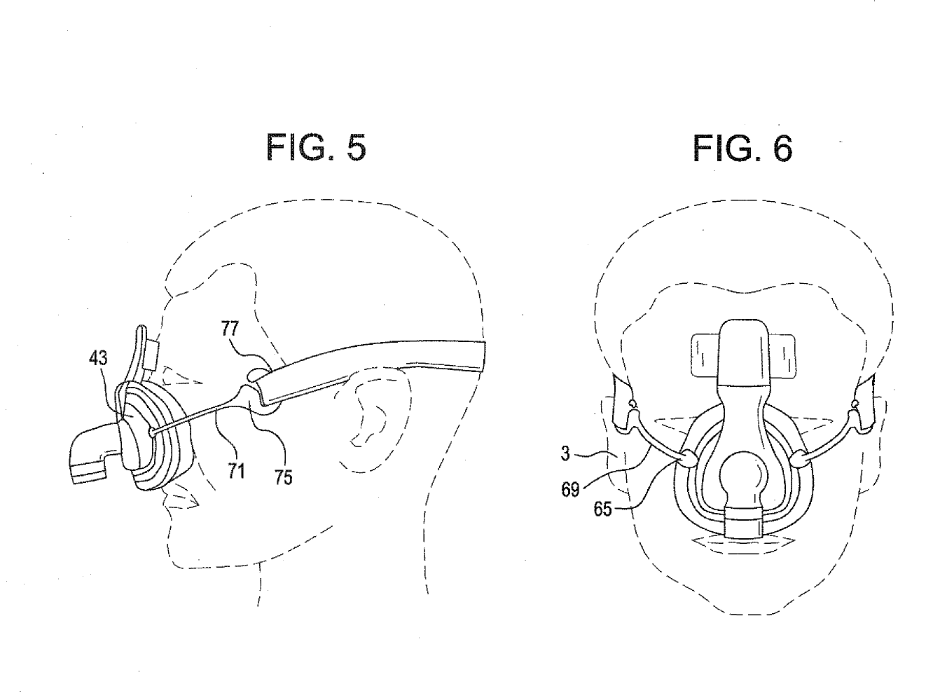

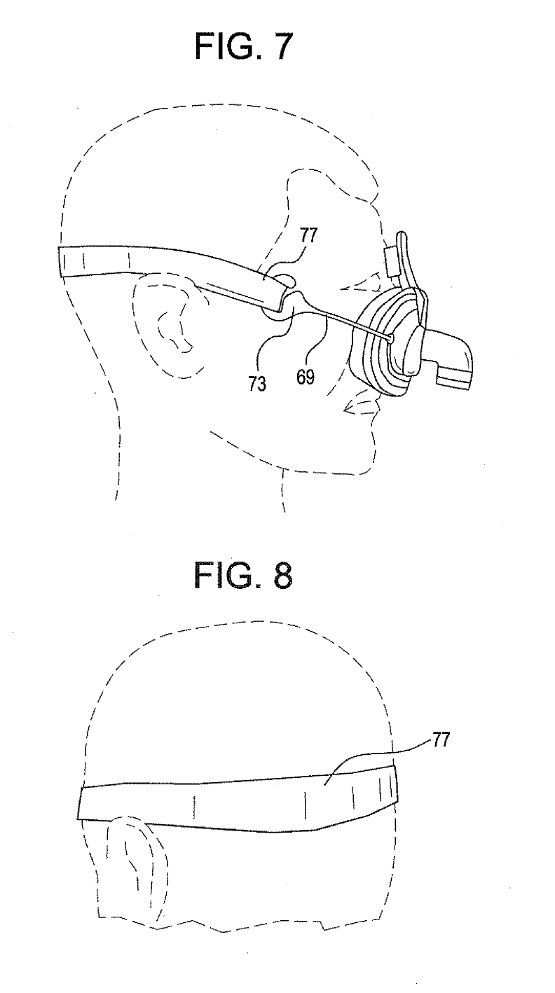

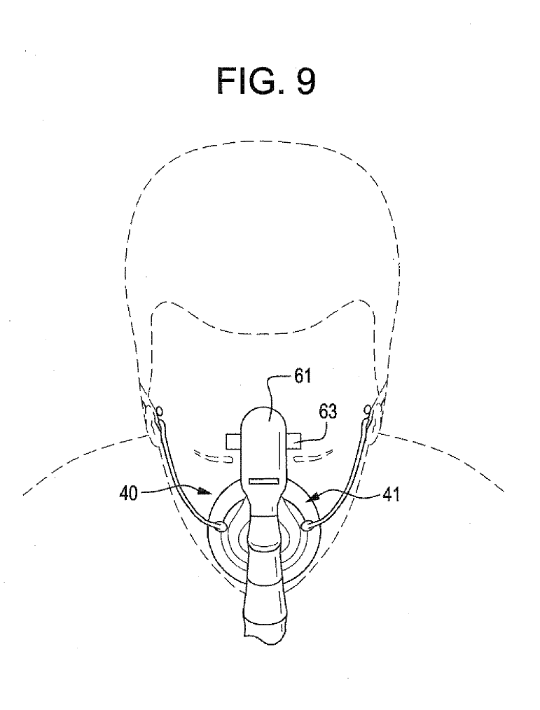

With reference to FIGS. 3-9, the inventive breathing mask is generally designated by the reference numeral 40 and is seen to include a housing 41 in which a chamber 43 is defined. As seen in particular in FIGS. 5-7, the chamber 43 surrounds the nose of the wearer. The mask 40 includes a central forward facing opening 45 to which is affixed a generally L-shaped conduit 47 including a horizontal portion 49 and a vertical portion 51 coupled thereto in a manner allowing relative rotation and pivoting therebetween. The conduit 47 is connectable to a source of pressurized air (FIG. 4).

As seen in FIG. 4, the housing 41 is generally triangular in shape with the lower extent being generally horizontal at 53 and with two angled walls 55 and 57 joining at an apex 59 (FIG. 4). Emanating vertically from the apex 59 is a vertically disposed bracket 61 to which is attached a generally horizontal cushion 63 that is designed to engage the forehead 2 of the wearer 1.

The side walls 55 and 57 of the housing 41 include connection points 65 and 67 (FIG. 6) to which are attached strap portions 69 and 71, respectively. The connection points 65 and 67 are located at the horizontal axis of rotation of the mask to limit any pivoting about that axis that would tend to dislodge the face engaging portion 56 that peripherally seals the mask housing 41. As seen in FIGS. 5-7, the strap portions 69 and 71 each terminate distally from the housing 41 in a slot portion 73 and 75, respectively, that receives a loop of a wider elastic strap 77. As seen in particular in FIGS. 5, 7 and 8, the strap 77 extends around the rear of the user's head 1 above the ears 3 thereof.

As should be understood from FIGS. 3-9, the inventive breathing mask is as effective as prior art masks in facilitating supply of pressurized air to a wearer through their nose. However, the inventive breathing mask is superior to prior art designs because it dramatically simplifies the structure of such breathing masks, requiring only a single strap over the ears of the wearer, while that single strip is positioned in such a manner that the mask is securely placed over the nose without allowing any peripheral leakage. This is ensured by the flexible face engaging portion 56 (FIG. 4) that conforms to the face 4 of the wearer and provides a tight seal completely around the periphery of the wearer's nose. With only a single strap 77 connected to the housing 41 by only two strap portions 69 and 71, the inventive mask 40 is much less detactable by the wearer while sleeping, thereby making it more comfortable to sleep while wearing the mask. By the same token, when a wearer wakes up in the middle of the night for any reason, it is more easy to remove the mask and replace it without completely awakening, thereby facilitating a greater ability to go back to sleep.

In the preferred embodiment of the present invention, the housing 41 is made of a molded plastic while the face engaging portion 56 is made of a soft plastic or rubber. The strap portions 69 and 71 may be made of any plastic or metal, and the strap 77 is preferably made of an elastic material. The cushioning member 63 is preferably made of any desired foam rubber or other foamed material and may suitably be affixed to the bracket 61 using an adhesive or perhaps a rivet or other desired attachment means.

The coupling between the portions 49 and 51 of the inlet conduit 47 may comprise rotating and/or universal joints. The conduit 47 may be made of any suitable plastic or metal.

As such, an invention has been disclosed in terms of a preferred embodiment thereof which fulfills each and every one of the objects as set forth hereinabove, and provides a new and useful improved sleep apnea breathing mask of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. An improved breathing mask comprising:
a) a housing defining an internal chamber sized to surround a nose of a wearer;
b) a conduit connecting said chamber with a source of air;
c) a single strap attached to sides of said housing and configured to extend around a head of a wearer; and
d) a forehead pad connected to said housing.

2. The mask of claim 1, wherein said housing includes a resilient periphery configured to create a seal against a face of a wearer.

3. The mask of claim 1, wherein said forehead pad is connected to said housing via a bracket.

4. The mask of claim 3, wherein said bracket extends vertically above said housing.

5. The mask of claim 4, wherein said pad is horizontally disposed.

6. The mask of claim 3, wherein said housing is generally triangular, said bracket emanating from a top apex of said housing.

7. The mask of claim 4, wherein said housing is generally triangular, said bracket emanating from a top apex of said housing.

8. The mask of claim 1, wherein said single strap includes first and second strap portions each attached at one end to said housing and each having a slot portion remote from said housing.

9. The mask of claim 1, wherein said source of air comprises a source of pressurized air.

10. The mask of claim 1, wherein said conduit is L-shaped.

11. The mask of claim 10, wherein said L-shaped conduit comprises two conduit legs coupled together at a movable joint.

12. The mask of claim 1, wherein said pad is horizontally disposed.

13. The mask of claim 1, wherein said housing is made of plastic.

14. The mask of claim 2, wherein said resilient periphery is made of rubber.

15. The mask of claim 6, wherein said housing is made of plastic.

16. An improved breathing mask comprising:
a) a generally triangular housing defining an internal chamber sized to surround a nose of a wearer;
b) an L-shaped conduit connecting said chamber with a source of pressurized air;
c) a single strap attached to sides of said housing and configured to extend around a head of a wearer; and
d) a horizontally disposed forehead pad connected to said housing via a bracket.

17. The mask of claim 16, wherein said housing includes a resilient periphery configured to create a seal against a face of a wearer.

18. The mask of claim 16, wherein said bracket extends vertically above said housing.

19. The mask of claim 16, wherein said single strap includes first and second strap portions each attached at one end to said housing and each having a slot portion remote from said housing, and an elastic strap connected to said slot portions.

20. The mask of claim 16, wherein said housing is made of plastic and said resilient periphery is made of rubber.
